(19) United States
(12) Patent Application Publication
(10) Pub. No.: US 2024/0398777 A1
(43) Pub. Date: Dec. 5, 2024

(54) METHOD OF TREATING SYMPTOMS OF PRADER-WILLI SYNDROME

(71) Applicant: HARMONY BIOSCIENCES, LLC, Plymouth Meeting, PA (US)

(72) Inventors: Kumaraswamy Budur, Chicago, IL (US); Krystle Davis Rapchak, Chicago, IL (US); Jeffrey Marc Dayno, Ambler, PA (US); Eric David Bauer, Wilmette, IL (US)

(21) Appl. No.: 18/679,624

(22) Filed: May 31, 2024

Related U.S. Application Data
(60) Provisional application No. 63/505,484, filed on Jun. 1, 2023.
(60) Provisional application No. 63/513,044, filed on Jul. 11, 2023.
(60) Provisional application No. 63/588,156, filed on Oct. 5, 2023.

(51) Int. Cl. A61K 31/4453; A61P 25/26

(52) U.S. Cl. CPC A61K 31/4453; A61P 25/26

(57) ABSTRACT

The present disclosure relates generally methods of treating symptoms of Prader-Willi syndrome (PWS), including excessive daytime sleepiness (EDS), hyperphagia, and/or behavioral problems, comprising administering to a subject with PWS a therapeutically effective amount of pitolisant or a pharmaceutically acceptable salt thereof.

### RELATED APPLICATIONS

This application claims the benefit of U.S. Provisional Application No. 63/505,484, filed on Jun. 1, 2023, U.S. Provisional Application No. 63/513,044, filed on Jul. 11, 2023, and U.S. Provisional Application No. 63/588,156, filed on Oct. 5, 2023, the contents of which are each incorporated herein by reference in their entireties.

### BACKGROUND

Prader-Willi Syndrome (PWS) is a genetic neurodevelopmental disorder characterized by symptoms that include hyperphagia (excessive eating), early onset childhood obesity, excessive daytime sleepiness (EDS), behavioral problems, and cognitive impairment. (See Cassidy S B, et al. Genet. Med. 2012; 14 (1): 10-26). EDS is a common symptom in subjects with PWS but is often underrecognized, and there is an association between EDS and emotional/behavioral disturbances in children and young people with PWS. (See Camfferman D, et al. Sleep Med. Rev. 2008; 12 (1): 65-75; see also Ghergan A, et al. Sleep 2017; 40 (12): zsx162). EDS often begins in early childhood and may continue across a person's lifespan. (See Camfferman, supra; see also Ghergan, supra). As a result, EDS can interfere with learning, adversely affect daily functioning and performance, and contribute to behavioral disorders. (See Ghergan, supra; see also Duis J, et al. J. Clin. Sleep Med. 2022; 18 (6): 1687-1696). Behavioral problems are a significant issue for many people living with PWS, particularly temper outbursts, social isolation, impulsivity, obsessive-compulsive tendencies, and perseveration, among others, which can significantly reduce the quality of life of the patient and their caregiver or family. (See U.S. Food and Drug Administration (FDA) Externally-Led Patient Focused Drug Development Meeting (EL-PFDD) Voice of the Patient Report, Prader Willi Syndrome, Apr. 22, 2024).

Many of the clinical manifestations of PWS are related to abnormalities in the development and function of the hypothalamus, which is a crucial region of the brain for regulating endocrine function, body temperature, and, importantly, sleep-wake timing and stability. (See Tauber M. et al. Lancet Diabetes Endocrinol. 2021; 9 (4): 235-246. See also Ono D, Yamanaka A. Neurosci. Res. 2017; 118:74-81). The neurotransmitter histamine is involved in a wide range of physiological functions, including regulation of the sleep-wake cycle, arousal, cognition, and memory. (See Scammell T, et al. Sleep 2019; 42 (1): zsy 183; see also Tashiro M, et al. Life Sci. 2002; 72 (4-5): 409-414).

There is a need for new treatments of disorders suffered by subjects with PWS, such as EDS.

### SUMMARY

The present disclosure relates generally to a method of treating excessive daytime sleepiness (EDS) in a subject with Prader-Willi syndrome (PWS), comprising administering to a subject with PWS a therapeutically effective amount of pitolisant or a pharmaceutically acceptable salt thereof (e.g., pitolisant hydrochloride, e.g., pitolisant monohydrochloride).

Also disclosed herein are methods of reducing hyperphagia in a subject with PWS, comprising administering to a subject with PWS a therapeutically effective amount of pitolisant or a pharmaceutically acceptable salt thereof (e.g., pitolisant hydrochloride, e.g., pitolisant monohydrochloride).

Also disclosed herein are methods of reducing or improving behavioral problems in a subject with PWS (e.g., irritability, social withdrawal, hyperactivity/noncompliance, inappropriate speech, or stereotypic behavior), comprising administering to a subject with PWS a therapeutically effective amount of pitolisant or a pharmaceutically acceptable salt thereof (e.g., pitolisant hydrochloride, e.g., pitolisant monohydrochloride).

Pitolisant is a histamine 3 receptor antagonist/inverse agonist that can enhance histamine release in the brain. (See Scammell, supra). A drug known as WAKIX® that contains pitolisant monohydrochloride has been approved by the U.S. Food and Drug Administration (FDA) for use in patients with narcolepsy. (See WAKIX Prescribing information. Harmony Biosciences, LLC; 2022)

A method disclosed herein may comprise administering to a subject with PWS pitolisant or a pharmaceutically acceptable salt thereof (e.g., pitolisant monohydrochloride), such as WAKIX®.

The methods disclosed herein can comprise administering to a subject with PWS a dose of about 1 mg to about 50 mg of pitolisant (mass expressed in terms of freebase pitolisant), e.g., about 4 mg to about 45 mg, e.g., about 4.5 mg, about 9 mg, about 18 mg, about 22 mg, about 36 mg, or about 35 mg pitolisant freebase. For example, a method disclosed herein may comprise administering to the subject about 7 mg to about 11 mg, about 11 mg to about 15 mg, about 16 mg to about 20 mg, about 25 mg to about 29 mg, about 34 mg to about 38 mg, e.g., about 4.5 mg, about 9 mg, about 18 mg, about 22 mg, about 36 mg, or about 45 mg pitolisant, wherein mass is based on the freebase form of pitolisant. For example, the dose can be about 4.45 mg, about 8.9 mg, about 17.8 mg, about 22.25 mg, about 35.6 mg, or about 44.5 mg pitolisant freebase.

It will be understood that when a pharmaceutically acceptable salt of pitolisant is administered, such as pitolisant monohydrochloride, the amount by weight of the salt will be larger than the equivalent amount of freebase. For example, a 5 mg dose of pitolisant monohydrochloride is equivalent to a 4.45 mg dose of pitolisant freebase, and a 20 mg dose of pitolisant monohydrochloride is equivalent to a 17.8 mg dose of pitolisant freebase. As such, a method disclosed herein may comprise administering to a subject an amount of pitolisant monohydrochloride that is equivalent to about 1 mg to about 50 mg of pitolisant freebase.

A method disclosed herein may comprise titrating the dose, e.g., administering an initial dose of pitolisant or a pharmaceutically acceptable salt thereof to a subject with PWS, and then increasing the dose (e.g., doubling the dose) each week for a period of 1-3 weeks after the initial dose is administered, to attain a maintenance dose. For example, an initial dose may be given to the subject for one week, then increased (e.g., doubled) for the second week. In some aspects, an initial dose may be given to the subject for one week, then increased (e.g., doubled) for the second week, then increased (e.g., doubled) again for the third week. In some aspects, an initial dose may be given to the subject for one week, then increased (e.g., doubled) for the second week, then increased (e.g., doubled) again for the third week, and then increased (e.g., doubled) yet again for the fourth week.

The subject can be an adult, child, or adolescent. The subject may have a weight of less than or equal to 40 kg, or greater than 40 kg and less than or equal to 80 kg, or greater than 80 kg.

The age or weight of the subject may be used to determine an appropriate dose. For example, in one aspect a subject with PWS that weighs greater than 40 kg and less than or equal to 80 kg (e.g., an adult or adolescent) may receive an initial dose of about 4.45 mg or about 8.9 mg, optionally increasing to about 8.9 mg or about 17.8 mg at about 1 week after the initial dose is administered, and optionally increasing to a maintenance dose of about 17.8 mg or about 35.6 mg at about 2 weeks after the initial dose is administered. Alternatively, a subject with PWS that weighs greater than 80 kg (e.g., an adult) may be administered an initial dose of about 4.45 mg or about 8.9 mg, optionally increasing to about 8.9 mg or about 17.8 mg at about 1 week after the initial dose is administered, optionally increasing to about 17.8 mg or about 35.6 mg at about 2 weeks after the initial dose is administered, and optionally increasing to a maintenance dose of about 22.25 mg or about 44.5 mg at about 3 weeks after the initial dose is administered. Further, in another aspect wherein the subject with PWS weighs less than or equal to 40 kg (e.g., a child or adolescent), the subject can be administered an initial dose of about 4.45 mg or about 8.9 mg, optionally increasing to a maintenance dose of about 8.9 mg or about 17.8 mg at about 1 week after the initial dose is administered.

In some aspects, the present disclosure relates to a method of treating excessive daytime sleepiness (EDS) in a subject with Prader-Willi syndrome (PWS). The method may comprise administering to a subject with PWS a therapeutically effective amount of pitolisant or a pharmaceutically acceptable salt thereof. The subject can be administered pitolisant monohydrochloride, e.g., WAKIX®.

A therapeutically effective amount of pitolisant may comprises a dose of about 1 mg to about 50 mg of pitolisant, e.g., about 4 mg to about 45 mg, e.g., about 4.5 mg, about 9 mg, about 18 mg, about 22 mg, about 36 mg, or about 45 mg pitolisant, wherein mass is based on the freebase form of pitolisant. For example, the therapeutically effective amount may comprise a dose of about 4.45 mg, about 8.9 mg, about 17.8 mg, about 22.25 mg, about 35.6 mg, or about 44.5 mg pitolisant, wherein mass is based on the freebase form of pitolisant.

A method disclosed herein may comprise administering an initial dose of pitolisant to the subject that is optionally increased (e.g., doubled) each week for a period of 1-3 weeks after the initial dose is administered, to attain a maintenance dose.

For example, a subject with PWS that has a body weight of greater than 40 kg and less than or equal to 80 kg may be administered an initial dose of pitolisant of about 4.45 mg or about 8.9 mg, optionally increasing to about 8.9 mg or about 17.8 mg at about 1 week after the initial dose is administered, and optionally increasing to a maintenance dose of about 17.8 mg or about 35.6 mg at about 2 weeks after the initial dose is administered.

In another example, a subject with PWS that weighs greater than 80 kg may be administered an initial dose of pitolisant of about 4.45 mg or about 8.9 mg, optionally increasing to about 8.9 mg or about 17.8 mg at about 1 week after the initial dose is administered, optionally increasing to about 17.8 mg or about 35.6 mg at about 2 weeks after the initial dose is administered, and optionally increasing to a maintenance dose of about 22.25 mg or about 44.5 mg at about 3 weeks after the initial dose is administered.

In another example, a subject with PWS that weighs less than or equal to 40 kg may be administered an initial dose of pitolisant of about 4.45 mg or about 8.9 mg, optionally increasing to a maintenance dose of about 8.9 mg or about 17.8 mg at about 1 week after the initial dose is administered.

The subject with PWS can be an adult (e.g., 18 to 65 years old), an adolescent (e.g., 12 years or older, and younger than 18 years old), or a child (e.g., 6 years or older, and younger than 12 years old).

The methods disclosed herein can improve the symptoms of a subject suffering with PWS. For example, methods disclosed herein can improve the subject's propensity for sleep, e.g., as determined by a validated measurement for sleep propensity (e.g., Epworth Sleepiness Scale for Children and Adolescents (ESSCHAD). For example, a method disclosed herein may lead to a decrease of 2 or more points on the ESS-CHAD, e.g., a decrease of 3, 4, 5, 6, or more points. Methods disclosed herein can reduce the subject's Caregiver Global Impression of Severity (CaGI-S) score, e.g., by 0.5, 0.6, 0.7, 0.8, 0.9, 1.0, 1.1, 1.2, 1.3, 1.4. 1.5, 1.6. 1.7, 1.8, 1.9, 2.0, or more, e.g., after a period of 11 or more weeks of treatment with pitolisant.

Methods disclosed herein can reduce hyperphagia in a subject with PWS, which can be determined by a validated measurement for hyperphagia, such as the Hyperphagia Questionnaire for Clinical Trials (HQ-CT), e.g., determined by a reduction in HQ-CT score of 1.0, 1.2, 1.4, 1.6, 1.8. 2.0, 2.2, 2.4, 2.6, 2.8, 3.0, 3.2, 3.4, or more, e.g., after a period of 11 or more weeks of treatment with pitolisant.

Methods disclosed herein can reduce or improve behavioral problems experienced by subjects with PWS. For example, methods disclosed herein can reduce irritability in the subject, e.g., as determined by a validated measurement for irritability (e.g., Aberrant Behavior Checklist-2 (ABC-2), e.g., by a reduction in ABC-2 score for irritability of 0.2, 0.4, 0.6, 0.8, 1.0, 1.5, 2.0, 2.5, 3.0, 3.5, 4.0, 4.5, 5.0, 5.5, 6.0, 6.5, 7.0, 7.5, 8.0, or more, e.g., after a period of 11 or more weeks of treatment with pitolisant). Methods disclosed herein may also reduce social withdrawal in the subject, e.g., as determined by a validated measurement for social withdrawal (e.g., ABC-2, e.g., by a reduction in ABC-2 score for social withdrawal of 0.2, 0.4, 0.6, 0.8, 1.0, 1.5, 2.0, 2.5, 3.0, 3.5, 4.0, 4.5, 5.0, 5.5, 6.0, 6.5, 7.0, 7.5, 8.0, or more, e.g., after a period of 11 or more weeks of treatment with pitolisant). Methods disclosed herein can reduce hyperactivity/noncompliance in the subject, e.g., as determined by a validated measurement for hyperactivity/noncompliance (e.g., ABC-2, e.g., by a reduction in ABC-2 score for social hyperactivity/noncompliance of 0.2, 0.4, 0.6, 0.8, 1.0, 1.5, 2.0, 2.5, 3.0, 3.5, 4.0, 4.5, 5.0, 5.5, 6.0, 6.5, 7.0, 7.5, 8.0, or more, e.g., after a period of 11 or more weeks of treatment with pitolisant). Methods disclosed herein can reduce inappropriate speech in the subject, e.g., as determined by a validated measurement for inappropriate speech (e.g., ABC-2, e.g., by a reduction in ABC-2 score for inappropriate speech of 0.2, 0.4, 0.6, 0.8, 1.0, 1.5, 2.0, 2.5, 3.0, 3.5, 4.0, 4.5, 5.0, 5.5, 6.0, 6.5, 7.0, 7.5, 8.0, or more, e.g., after a period of 11 or more weeks after being administered pitolisant). Methods disclosed herein can reduce stereotypic behavior (e.g., behavior stereotypic for PWS) in the subject, e.g., as determined by a validated measurement for stereotypic behavior (e.g., ABC-2, e.g., by a reduction in ABC-2 score for stereotypic behavior of 0.2, 0.4, 0.6, 0.8, 1.0, 1.5, 2.0, 2.5, 3.0, 3.5, 4.0, 4.5, 5.0, 5.5, 6.0, 6.5, 7.0, 7.5, 8.0, or more, e.g., after a period of 11 or more weeks of treatment with pitolisant).

### BRIEF DESCRIPTION OF DRAWINGS

FIG. 1 shows a diagram depicting the study design of a phase 2 proof-of-concept study for treating EDS in subjects with PWS.

FIG. 2 shows a bar graph comparing the mean change from baseline at week 11 of ESS-CHAD (parent/caregiver version) scores in subjects that received high dose pitolisant, low dose pitolisant, or placebo.

FIG. 3 shows a bar graph comparing the ESS-CHAD (parent/caregiver version) response rates at week 11 in subjects that received high dose pitolisant, low dose pitolisant, pooled dose groups, and placebo.

FIG. 4 shows a bar graph comparing the mean change from baseline at week 11 of Caregiver Global Impression of Severity (CaGI-S) scores in subjects that received higher-dose pitolisant, lower-dose pitolisant, and placebo.

FIG. 5 shows a bar graph comparing the mean change from baseline at week 11 of Hyperphagia Questionnaire for Clinical Trials (HQ-CT) scores in subjects that received higher-dose pitolisant, lower-dose pitolisant, and placebo.

FIG. 6 shows a bar graph comparing the mean change from baseline at week 11 of Aberrant Behavior Checklist-2 (ABC-2) scores in child subjects (aged 6 to <12 years) that received higher-dose pitolisant, lower-dose pitolisant, and placebo.

### DETAILED DESCRIPTION

Disclosed herein are methods of treating symptoms of Prader-Willi syndrome (PWS) comprising administering to a subject with PWS a therapeutically effective amount of pitolisant or a pharmaceutically acceptable salt thereof. In particular, disclosed herein are methods of reducing excessive daytime sleepiness (EDS), hyperphagia, and/or behavioral problems (e.g., irritability, social withdrawal, hyperactivity/noncompliance, inappropriate speech, or stereotypic behavior) in a subject with PWS, using pitolisant or a pharmaceutically acceptable salt thereof. A method disclosed herein may comprise administering WAKIX® to the subject with PWS.

The methods disclosed herein surprisingly can treat these symptoms of PWS that currently have no remedy, which is significant as those symptoms cause significant loss in quality of life of subjects living with PWS, as well as their caregivers and families, particularly EDS, hyperphagia, and behavioral problems. The efficacy of the methods disclosed herein has been demonstrated in the present disclosure with clinical studies using validated measurements such as Epworth Sleepiness Scale for Children and Adolescents (ESS-CHAD), Caregiver Global Impression of Severity (CaGI-S) for EDS, Aberrant Behavior Checklist-2 (ABC-2), Hyperphagia Questionnaire for Clinical Trials (HQ-CT) and the Food Safe Zone Questionnaire (FSZQ).

### Definitions

The articles “a” and “an” are used herein to refer to one or more than one (i.e., to at least one) of the grammatical object of the article. By way of example, “an element” means one element or more than one element.

The term “about” when referring to a measurable value such as an amount, a temporal duration, and the like, is meant to encompass variations of ±20% or less, or in some instances ±15% or less, or in some instances ±10% or less, or in some instances ±5% or less, or in some instances ±1% or less, or in some instances ±0.1% or less, from the specified value, as such variations are appropriate.

The phrase “and/or” as used herein should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B,” when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

The terms “administer,” “administering,” or “administration,” as used herein refer to implanting, absorbing, ingesting, injecting, inhaling, or otherwise introducing a compound, dosage form, or pharmaceutical composition.

The terms “comprise,” “comprises,” and “comprising” are used herein in a non-exclusive sense, except where the context requires otherwise. Likewise, the term “include” and its grammatical variants are intended to be non-limiting, such that recitation of items in a list is not to the exclusion of other like items that can be substituted or added to the listed items.

The term “effective amount” or a “therapeutically effective amount” as used herein refers to an amount of a compound, a dosage form, or a pharmaceutical composition, described herein which is sufficient to achieve a desired result under the conditions of administration. For example, an effective amount of a compound, dosage form, or pharmaceutical composition disclosed herein for treating excessive sleep disorder (EDS), e.g., in a subject with Prader-Willi Syndrome, is an amount that can reduce the effects of the EDS, and/or reduce or eliminate the severity of a symptom associated with the EDS.

The term “pharmaceutically acceptable excipient” as used herein refers to a non-toxic material that may be formulated with a compound disclosed herein to provide a pharmaceutical composition. Preferably, the pharmaceutically acceptable excipient is inert and does not interfere with the pharmacological activity of a compound which it is formulated with. Pharmaceutically acceptable excipients useful in the manufacture of the pharmaceutical compositions disclosed herein are any of those well known in the art, and include without limitation, diluents, dispersing agents, granulating agents, surface active agents, emulsifiers, disintegrating agents (sometimes referred to herein as disintegrants), binding agents (sometimes referred to herein as binders), preservatives, buffering agents (sometimes referred to herein as buffers), lubricating agents (sometimes referred to herein as lubricants), glidants, adjuvants, fillers, wetting agents, suspending agents, solvents, dispersion media, ion exchangers, salts, electrolytes, waxes, and/or oils, and the like.

The term “pharmaceutically acceptable salt” as used herein refers to a salt of a compound prepared with relatively nontoxic acids or bases, depending on the particular substituents found on the respective compound. When compounds relating to the present disclosure contain relatively basic functionalities, acid addition salts can be obtained by contacting the neutral form of such compounds with a sufficient amount of the desired acid, either neat or in a suitable solvent (e.g., an inert solvent). For example, the neutral form of pitolisant (the freebase) may be contacted with hydrochloric acid (e.g., gaseous hydrochloric acid) to provide pitolisant monohydrochloride. Examples of pharmaceutically acceptable acid addition salts include those derived from inorganic acids like hydrochloric acid, hydrobromic acid, nitric acid, carbonic acid, monohydrogencarbonic acid, phosphoric acid, monohydrogenphosphoric acid, dihydrogenphosphoric acid, sulfuric acid, monohydrogensulfuric acid, hydriodic acid, or phosphorous acids and the like, as well as the salts derived from organic acids like acetic acid, propionic acid, isobutyric acid, maleic acid, malonic acid, benzoic acid, succinic acid, suberic acid, fumaric acid, lactic acid, mandelic acid, pamoic acid, phthalic acid, benzenesulfonic acid, p-toluenesulfonic acid, citric acid, tartaric acid, methanesulfonic acid, oxalic acid, and the like. Also included are salts of amino acids such as arginate and the like, and salts of organic acids like glucuronic or galacturonic acids and the like. Other pharmaceutically acceptable salts known to those of skill in the art are suitable for pharmaceutical compositions relating to the present disclosure.

The term “solvate” as used herein refers to a form of a compound that is associated with a solvent, usually by a solvolysis reaction. This physical association may include hydrogen bonding. Conventional solvents include water, methanol, ethanol, acetic acid, dimethyl sulfoxide (DMSO), tetrahydrofuran (THF), diethyl ether, and the like. Compounds of the present disclosure may be prepared, e.g., in crystalline form, and may be solvated. Suitable solvates include pharmaceutically acceptable solvates and further include both stoichiometric solvates and non-stoichiometric solvates. In certain instances, the solvate will be capable of isolation, for example, when one or more solvent molecules are incorporated in the crystal lattice of a crystalline solid. “Solvate” encompasses both solution-phase and isolable solvates. Representative solvates include hydrates, ethanolates, and methanolates.

The term “hydrate” as used herein refers to a compound which is associated with water. Typically, the number of the water molecules contained in a hydrate of a compound is in a definite ratio to the number of the compound molecules in the hydrate. Therefore, a hydrate of a compound may be represented, for example, by the general formula R·xH₂O, wherein R is the compound and wherein x is a number greater than 0. A given compound may form more than one type of hydrate, including, e.g., monohydrates (x is 1), lower hydrates (x is a number greater than 0 and smaller than 1, e.g., hemihydrates (R·0.5H₂O)), and polyhydrates (x is a number greater than 1, e.g., dihydrates (R·2H₂O) and hexahydrates (R·6H₂O)).

The term “subject” as used herein refers to any animal, such as any mammal, including but not limited to, humans, non-human primates, rodents, dogs, and the like. Non-human primates include chimpanzees, cynomolgus monkeys, spider monkeys, and macaques (e.g., Rhesus). Rodents include mice, rats, woodchucks, ferrets, rabbits, and hamsters. Domestic and game animals include cows, horses, pigs, deer, bison, buffalo, feline species (e.g., domestic cat), canine species (e.g., dog, fox, wolf), avian species, and fish. In some embodiments, the subject is a mammal (e.g., a human, a rat, or a mouse). The subject can be male or female. The subject may be of any age, including a child (e.g., 6 to less than 12 years of age), an adolescent (e.g., 12 to less than 18 years of age), or an adult (e.g., 18 years or older, e.g., 18 to 65 years of age). The subject may be an elderly human subject (e.g., 65 years or older), a human subject that is not elderly (e.g., less than 65 years old), or a human pediatric subject (e.g., less than 18 years old). In preferred aspects, the subject is a human.

As used herein, the terms “treat,” “treatment,” “treating,” or grammatically related terms, refer to a method of reducing the effects of a disease or disorder. As is readily appreciated in the art, full eradication of the disease, disorder, or symptoms thereof is preferred but not a requirement for treatment. Desirable effects of treatment include, but are not limited to, preventing occurrence or recurrence of the disease or disorder, alleviation of symptoms, diminishment of any direct or indirect pathological consequences of the disease or disorder, or other improvement of any sign, symptom, or consequence of the disease or disorder, such as prolonged survival, less morbidity, and/or a lessening of side effects.

Throughout this disclosure, various embodiments can be presented in a range format (e.g., from X-Y). It should be understood that the description in range format is merely for convenience and brevity and should not be construed as an inflexible limitation on the scope of the present disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 5, from 1 to 4, from 1 to 3, from 2 to 6, from 2 to 4, from 3 to 6, etc., as well as individual numbers within that range, e.g., 1, 2, 2.8, 3, 3.6, 4, 5, 5.4, and 6. As another example, a range such as 95-99% includes 95%, 96%, 97%, 98%, or 99% and all subranges such as 96-99%, 96-98%, 96-97%, 97-99%, 97-98%, etc. This applies regardless of the breadth of the range

All publications (e.g., scientific journal articles, patent publications, and the like) cited in this disclosure are incorporated by reference in their entirety. To the extent the material incorporated by reference contradicts or is inconsistent with this specification, the specification will supersede any such material. The citation of any references herein is not an admission that such references are prior art to the present disclosure. Various terms relating to aspects of the description are used throughout the specification and claims. Such terms are to be given their ordinary meaning in the art unless otherwise indicated. Other specifically defined terms are to be construed in a manner consistent with the definitions provided herein.

Compounds (e.g., pharmaceutically active agents) disclosed herein may also comprise one or more isotopic substitutions. For example, hydrogen (H) may be in any isotopic form, including ¹H, ²H (D or deuterium), ³H (T or tritium); carbon (C) may be in any isotopic form, including ¹²C, ¹³C, and ¹⁴C; oxygen (O) may be in any isotopic form, including ¹⁶O and ¹⁸O; nitrogen (N) may be in any isotopic form, including ¹⁴N and ¹⁵N; and chlorine (Cl) may be in any isotopic form, including ³⁵Cl and ³⁷Cl.

Various embodiments of the compounds, dosage forms, pharmaceutical compositions, and methods herein are described in further detail below, and additional definitions may be provided throughout the specification.

### Examples

### Example 1. Phase 2, Randomized, Placebo-Controlled Study

### Methods

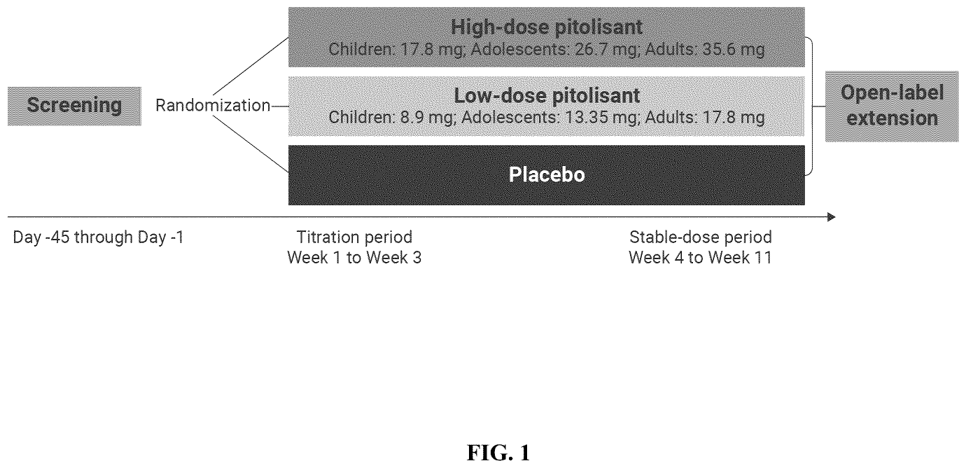

A study was carried out to evaluate the efficacy and safety of pitolisant for the treatment of EDS in subjects with PWS. This was a phase 2, randomized, placebo-controlled, proof-of-concept study. Children (aged 6 to <12 years), adolescents (aged 12 to <18 years), and adults (aged 18-65 years) with a confirmed diagnosis of PWS were randomly assigned to receive low-dose pitolisant (8.9 mg in children, 13.35 mg in adolescents, and 17.8 mg in adults) or high dose pitolisant (17.8 mg in children, 26.7 mg in adolescents, and 35.6 mg in adults), or placebo (see FIG. 1). All doses are expressed in terms of pitolisant freebase.

The 11-week double-blind treatment period consisted of a 3-week titration phase and an 8-week stable-dose phase.

The primary efficacy endpoint was the change from baseline to Week 11 in total score for the parent/caregiver version of the Epworth Sleepiness Scale for Children and Adolescents (ESS-CHAD), which is a validated measurement for sleep propensity. (See Janssen K C, et al. Sleep Med. 2017; 33:30-35; see also Patel V P, et al. J. Clin. Sleep Med. 2022; 18 (2): 485-496). A separate analysis was treatment response defined as ESS-CHAD score reduction of ≥3 points from baseline or Week 11 score of ≤10.

Secondary/exploratory endpoints included change from baseline in Caregiver Global Impression of Severity (CaGI-S) for EDS, behavioral disturbance (Aberrant Behavior Checklist-2 [ABC-2]), and hyperphagia (Hyperphagia Questionnaire for Clinical Trials [HQ-CT] in conjunction with the Food Safe Zone Questionnaire [FSZQ]). The primary time frame of interest was from baseline to Week 11.

The ABC-2 is a widely used and validated questionnaire that can assess changes in behavioral problems, and is particularly suitable for assessing behavioral changes in subjects with intellectual and developmental disabilities. The ABC-2 questions are subdivided into five domains (irritability, social withdrawal, hyperactivity/noncompliance, inappropriate speech, and stereotypic behavior). Generally, a reduction of 2 or more points in a domain of the ABC-2 is considered clinically meaningful. ABC-2 was used in this trial to determine if pitolisant improved or reduced behavioral problems experienced by subjects living with PWS.

### Results

A total of 65 patients were enrolled (see Table 1). 59 patients (90.8%) completed the study, and 58 of 59 completers opted to continue into an open-label extension phase. Mean scores on the ESS-CHAD at baseline (see Table 1) reflect EDS severe enough to warrant medical intervention (ESS-CHAD score ≥12 indicative of EDS).

TABLE 1
Demographic and Baseline Characteristics

| | | High-Dose | | Low-Dose | | | |
|---|---|---|---|---|---|---|---|
| | | Pitolisant | | Pitolisant | | Placebo | |
| Characteristic | | (n = 22) | | (n = 20) | | (n = 23) | |
| Age, years, mean (range) | | 13.1 | (6-28) | 12.1 | (6-24) | 11.9 | (6-23) |
| Age group, n (%) | | | | | | | |
| 6 to | <12 years | 11 | (50.0) | 12 | (60.0) | 11 | (47.8) |
| 12 to | <18 years | 6 | (27.3) | 4 | (20.0) | 9 | (39.1) |
| 18 to | 65 years | 5 | (22.7) | 4 | (20.0) | 3 | (13.0) |

| | | | | | | |
|---|---|---|---|---|---|---|
| Female, n (%) | 12 | (54.5) | 11 | (55.0) | 9 | (39.1) |
| Race, n (%) | | | | | | |
| White | 21 | (95.5) | 17 | (85.0) | 17 | (73.9) |
| Black or African American | 0 | (0) | 1 | (5.0) | 0 | (0) |
| Asian | 1 | (4.5) | 1 | (5.0) | 4 | (17.4) |
| Other | 0 | (0) | 1 | (5.0) | 2 | (8.7) |
| Baseline ESS-CHAD | 14.8 | (2.9) | 15.7 | (3.7) | 14.7 | (3.4) |
| score, mean (SD) | | | | | | |

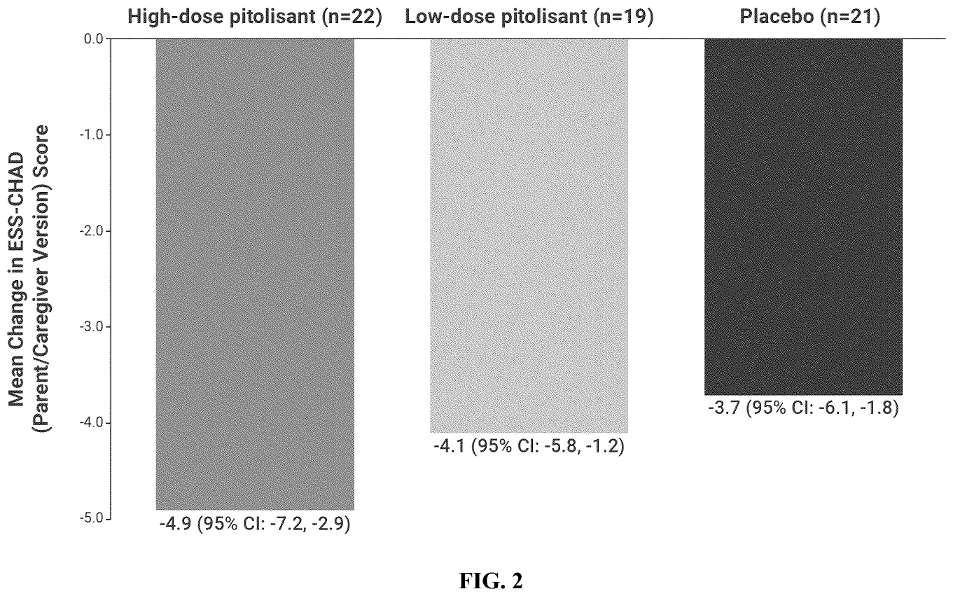

In the overall patient population, mean improvement from baseline in ESS-CHAD score was greater for both pitolisant dose groups compared with the placebo group at the end of treatment (see FIG. 2). There was an outlier in the placebo group—an adolescent with a baseline ESS-CHAD score of 24 (the scale maximum) and a Week 11 score of 4. A sensitivity analysis excluding this patient resulted in mean change scores on the ESS-CHAD that were more consistent across age groups (children, adolescents, adults) in the placebo group.

The strongest observed signal was in the youngest age group (6 to <12 years), which had the greatest number of patients (see Table 2). There was an unusually large placebo response in the adolescent age group (12 to <18 years), due to data from the single outlier. Because of the relatively small sample size, this resulted in an outsized impact on the magnitude of the placebo response not only in the adolescent subgroup but also in the overall study population.

TABLE 2
ESS-CHAD (Parent/Caregiver Version): Mean Change From Baseline
at Week 11, by Age Group*

| | | Mean Change (95% CI) From Baseline at Week 11 | | |
|---|---|---|---|---|
| | | High-Dose | Low-Dose | |
| Age Group | | Pitolisant | Pitolisant | Placebo |
| 6 to | <12 years | −5.5 | −3.7 | −2.1 |
| | | (−8.3, −2.9) | (−6.2, −1.0) | (−4.9, 0.7) |
| | | (n = 11) | (n = 12) | (n = 10) |
| 12 to | <18 years | −4.2 | −4.5 | −6.1 |
| | | (−7.2, 1.8) | (−12.4, −1.1) | (−9.6, −2.2) |
| | | (n = 6) | (n = 4) | (n = 8) |
| 18 to | 65 years | −4.4 | −5.0 | −2.3 |
| | | (−12.0, 3.0) | (−12.8, 7.2) | (−13.5, 6.7) |
| | | (n = 5) | (n = 3) | (n = 3) |

*Decrease of 2 points on the ESS-CHAD is considered clinically meaningful improvement. (See Maski K, et al. J. Clin. Sleep Med. 2021; 17(9): 1895-1945.)

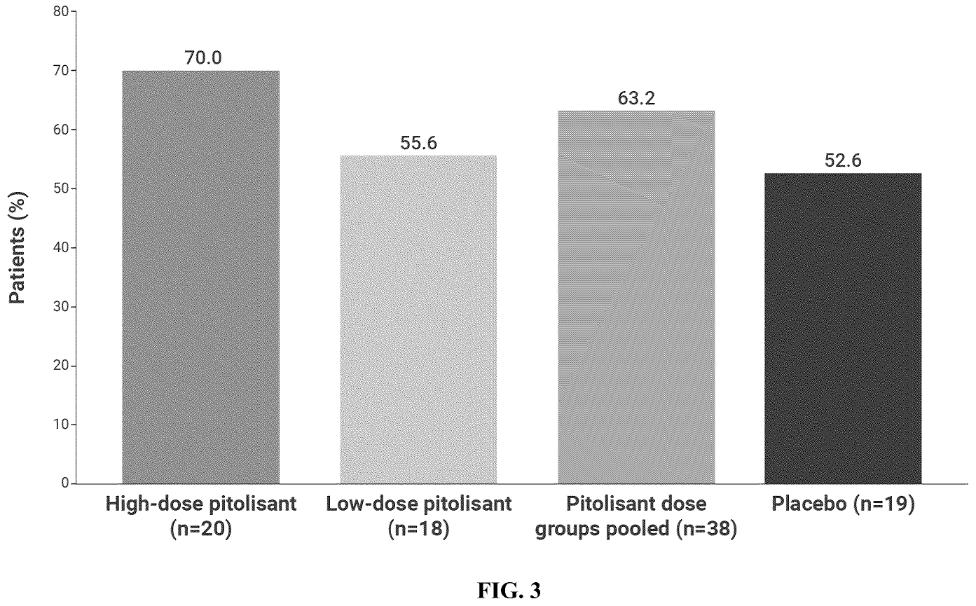

The response rate was higher for pitolisant versus placebo among study completers, driven primarily by the high-dose pitolisant group (see FIG. 3). Response was defined as ESS-CHAD (Parent/Caregiver Version) score reduction of ≥3 points from baseline or score of ≤10 at Week 11.

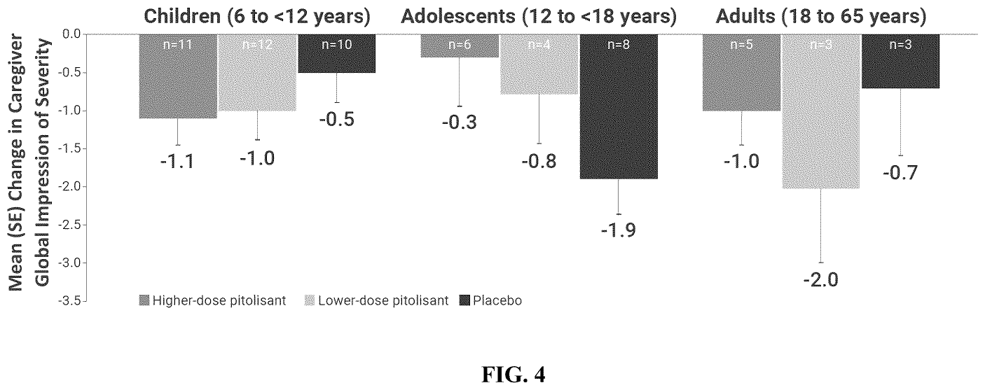

At week 11, reduction in CaGI-S score was greater for pitolisant compared with placebo in children (6 to <12 years) and adults (see FIG. 4). Mean (SD) change on the CaGI-S was −1.1 (1.1) in the higher-dose pitolisant group, −1.0 (1.2) in the lower-dose pitolisant group, and 0.5 (1.2) in the placebo group.

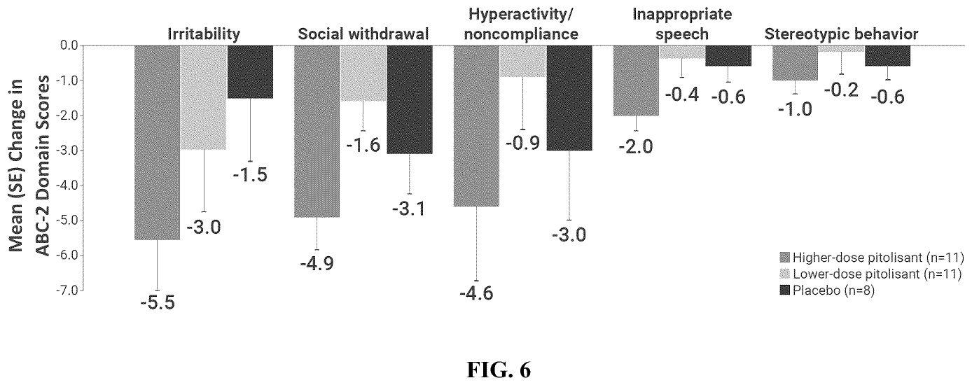

Pitolisant led to improvement in behavioral problems in the subjects. At week 11, mean reduction was greater for higher-dose pitolisant compared with placebo on all ABC-2 domains (irritability, social withdrawal, hyperactivity/noncompliance, inappropriate speech, stereotypic behavior) in children (6 to <12 years) (see FIG. 6). Results are also provided in Tables 3 and 4.

TABLE 3
ABC-2 mean change (SE) from baseline to end of treatment
(Week 11), in adolescents and adults (shown separately).

| | Adolescents (12 to <18 years) | | | | | | Adults (18 to 65 years) | | | | | |
|---|---|---|---|---|---|---|---|---|---|---|---|---|
| | Higher- | | Lower- | | Placebo | | Higher- | | Lower- | | Placebo | |
| ABC-2 Domain | dose (n = 6) | | dose (n = 4) | | (n = 8) | | dose (n = 5) | | dose (n = 3) | | (n = 3) | |
| Irritability | −5.0 | (3.2) | −4.8 | (2.8) | −7.6 | (5.0) | −2.8 | (1.5) | −3.0 | (3.1) | −3.0 | (6.0) |
| Social withdrawal | −3.7 | (1.2) | −3.3 | (1.3) | −2.5 | (2.0) | −2.4 | (1.2) | −1.0 | (1.2) | −2.3 | (5.6) |
| Hyperactivity/noncompliance | −3.8 | (1.5) | 0.0 | (2.3) | −4.0 | (4.2) | −5.4 | (1.8) | 1.0 | (5.2) | −1.0 | (3.1) |
| Inappropriate speech | −1.7 | (1.2) | −0.8 | (0.5) | −0.8 | (1.0) | −2.2 | (0.5) | 0.7 | (0.9) | 2.7 | (2.7) |
| Stereotypic behavior | −0.5 | (1.1) | 0.3 | (1.3) | −1.5 | (1.0) | −2.6 | (1.6) | −0.3 | (0.3) | −2.0 | (1.2) |

Data shown are mean (SE) change. ABC-2 is Aberrant Behavior Checklist-2.

TABLE 4
Summary of ABC-2 mean change from baseline to end of
treatment (Week 11) in all patient groups

| | Low Dose | High Dose | |
|---|---|---|---|
| | Pitolisant | Pitolisant | Placebo |
| ABC-2 Domain | (n = 18) | (n = 22) | (n = 19) |
| Irritability | −3.4 | −4.8 | −4.3 |
| Social withdrawal | −1.9 | −4.0 | −2.7 |
| Stereotypic behavior | −0.1 | −1.2 | −1.2 |
| Hyperactivity/noncompliance | −0.4 | −4.6 | −3.1 |
| Inappropriate speech | −0.3 | −2.0 | −0.2 |

A consistent reduction in behavioral problems across four of the five domains on the ABC-2 checklist was observed in the high dose group. Notably, clinically meaningful reductions of more than 4 points were seen in three of the domains including irritability and hyperactivity/noncompliance domains, which are behavioral problems that significantly affect people living with PWS. This clinically meaningful response across multiple domains of the ABC-2 was unexpected and surprising.

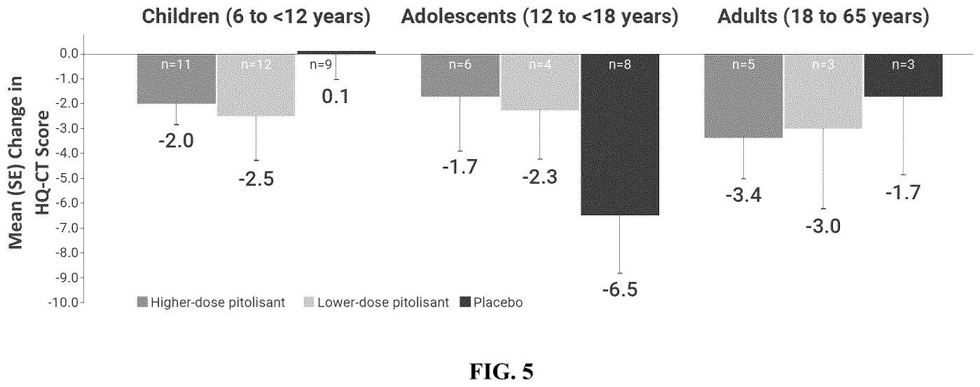

Pitolisant also reduced hyperphagia in the subjects. At week 11, reduction in the HQ-CT score was greater for pitolisant compared with placebo in children (6 to <12 years) and adults (18 to 65 years) (see FIG. 5). Mean (SD) HQ-CT score decreased with higher-dose pitolisant (−2.0 [2.8]) and lower-dose pitolisant (−2.5 [6.2]) and increased slightly with placebo (0.1 [3.4]).

Incidence of treatment-emergent adverse events (TEAEs) was similar for pitolisant and placebo. The most common adverse events were anxiety, irritability, and headache. The most common adverse events in pitolisant-treated patients (doses pooled) were irritability (17.4%) and anxiety (13.0%). The overall rate of adverse events was similar for pitolisant and placebo (see Table 5).

TABLE 5
Summary of Adverse Events

| | High-Dose | Low-Dose | |
|---|---|---|---|
| | Pitolisant | Pitolisant | Placebo |
| Adverse Events, n (%) | (n = 22) | (n = 20) | (n = 23) |
| Any TEAE | 11 (50.0) | 13 (65.0) | 15 (65.2) |
| Treatment-related* TEAEs | 4 (18.2) | 7 (35.0) | 7 (30.4) |
| Severe TEAEs | 0 | 0 | 0 |
| Serious TEAEs | 0 | 0 | 1 (4.3) |
| Most common TEAEs | 1 (4.5) | 4 (20.0) | 2 (8.7) |
| Anxiety | 1 (4.5) | 4 (20.0) | 1 (4.3) |
| Irritability | 2 (9.1) | 3 (15.0) | 2 (8.7) |
| Headache | | | |

*Considered by the investigator as possibly, probably, or definitely related to study drug.
TEAE = treatment-emergent adverse event.

### CONCLUSION

Pitolisant reduced EDS in patients with PWS; a dose-response relationship was observed, with higher-dose pitolisant showing a stronger efficacy signal. Specifically, improvement was observed on the primary endpoint, a change from baseline in the score on the parent/caregiver version of the Epworth Sleepiness Scale for Children and Adolescents (ESS-CHAD). The safety and tolerability of pitolisant in patients with PWS was consistent with the known safety profile for pitolisant; no new safety signals were observed. Pitolisant not only reduced EDS severity (as rated by caregivers), but also behavioral problems, and hyperphagia.

Based on the positive signals from this phase 2 study, a phase 3 clinical trial is being planned to further evaluate the safety and efficacy of pitolisant in the treatment of EDS in patients aged 6 years and older with a confirmed diagnosis of PWS.

### EQUIVALENTS

Those skilled in the art will recognize or be able to ascertain using no more than routine experimentation many equivalents to the specific embodiments disclosed herein. Those of ordinary skill in the art will appreciate that various changes or modifications to this description may be made without departing from the spirit or scope of the present disclosure, as defined in the following claims.

1. A method of treating excessive daytime sleepiness (EDS) in a subject with Prader-Willi syndrome (PWS), comprising administering to a subject with PWS a therapeutically effective amount of pitolisant or a pharmaceutically acceptable salt thereof.

2. The method of claim 1, wherein the subject is administered pitolisant monohydrochloride.

3. The method of claim 1, wherein the subject is administered WAKIX®.

4. The method of claim 1, wherein the therapeutically effective amount comprises a dose of about 1 mg to about 50 mg of pitolisant, wherein mass is based on the freebase form of pitolisant.

5. The method of claim 1, wherein the therapeutically effective amount comprises a dose of about 4.45 mg, about 8.9 mg, about 17.8 mg, about 22.25 mg, about 35.6 mg, or about 44.5 mg pitolisant, wherein mass is based on the freebase form of pitolisant.

6. The method of claim 1, wherein the method comprises administering an initial dose to the subject that is increased (e.g., doubled) each week for a period of 1-3 weeks after the initial dose is administered, to attain a maintenance dose.

7. The method of claim 1, wherein the subject with PWS weighs greater than 40 kg and less than or equal to 80 kg.

8. The method of claim 7, wherein the subject is administered an initial dose of about 4.45 mg or about 8.9 mg, optionally increasing to about 8.9 mg or about 17.8 mg at about 1 week after the initial dose is administered, and optionally increasing to a maintenance dose of about 17.8 mg or about 35.6 mg at about 2 weeks after the initial dose is administered.

9. The method of claim 1, wherein the subject with PWS weighs greater than 80 kg.

10. The method of claim 9, wherein the subject is administered an initial dose of about 4.45 mg or about 8.9 mg, optionally increasing to about 8.9 mg or about 17.8 mg at about 1 week after the initial dose is administered, optionally increasing to about 17.8 mg or about 35.6 mg at about 2 weeks after the initial dose is administered, and optionally increasing to a maintenance dose of about 22.25 mg or about 44.5 mg at about 3 weeks after the initial dose is administered.

11. The method of claim 1, wherein the subject with PWS weighs less than or equal to 40 kg.

12. The method of claim 11, wherein the subject is administered an initial dose of about 4.45 mg or about 8.9 mg, optionally increasing to a maintenance dose of about 8.9 mg or about 17.8 mg at about 1 week after the initial dose is administered.

13. The method of claim 1, wherein the subject with PWS is an adult.

14. The method of claim 1, wherein the subject with PWS is an adolescent.

15. The method of claim 1, wherein the subject with PWS is a child.

16. The method of claim 1, wherein the subject's propensity for sleep is improved, as determined by a validated measurement for sleep propensity.

17. The method of claim 1, wherein the subject's Caregiver Global Impression of Severity (CaGI-S) score is reduced.

18. The method of claim 1, further comprising reducing hyperphagia in the subject.

19. The method of claim 1, further comprising reducing irritability in the subject.

20. The method of claim 1, further comprising reducing social withdrawal in the subject.

21. The method of claim 1, further comprising reducing hyperactivity/noncompliance in the subject.

22. The method of claim 1, further comprising reducing inappropriate speech in the subject.

23. The method of claim 1, further comprising reducing stereotypic behavior in the subject.
